(19) United States
(12) Patent Application Publication
(10) Pub. No.: US 2006/0167469 A1
(43) Pub. Date: Jul. 27, 2006

(54) Suction controlled extraction device

(76) Inventor: Richard L. Arden, Farmington Hills, MI (US)

Correspondence Address: Arnold S Weintraub;The Weintraub Group, 32000 Northwestern Highway, Suite 240, Farmington Hills, MI, 48334, US

(21) Appl. No.: 10/511,643

(22) PCT Filed: Apr. 17, 2003
(86) PCT No.: PCT/US03/11925

§ 371 Date: Jan. 9, 2006

Related U.S. Application Data
(60) Provisional application No. 60/373,231, filed on Apr. 17, 2002.

(51) Int. Cl. A61F 11/00

(52) U.S. Cl. 606/109

(57) ABSTRACT

A suction controlled extraction device (10) for removing foreign bodies from the auditory canal or endonasal cavity of human includes a suction tube (12) having forward and rearward ends, the suction tube including first and second tube sections (14, 16), the rearward end of the second tube being connectible to a source of suction and the forward end of the first tube forming a frusto-conical insertion tip (22) that envelops and draws the foreign body thereinto when the suction is applied, a valve (17) for controlling and varying the pressure and suction drawn in the suction tube, and a magnifying lens (50) affixed to the suction tube to enable the user to see within the cavity and ensure that the insertion tip envelops the foreign object. The first tube is curvilinear, the opposite ends thereof angularly offset, and the frusto-conical insertion tip may be removable or integral with the first tube.

### CROSS-REFERENCE TO RELATED APPLICATION

This application claims the benefit of U. S. Provisional Application Ser. No. 60/373,231, filed Apr. 17, 2002, the disclosure of which is hereby incorporated by reference.

### BACKGROUND OF THE INVENTION

1. Field of the Invention:

The present invention pertains to medical devices. More particularly, the present invention concerns devices for extracting foreign objects from a body cavity or canal. Even more particularly, the present invention concerns suction devices for removing foreign bodies from the auditory canal or endonasal cavity or passage.

2. Description of Prior Art:

Foreign bodies located in the external auditory canal as well as the endonasal cavity continue to present unique challenges for the treating physician or other medical personnel. Reported incidences vary, but account for roughly 1/500 pediatric and 1/1500 adult attendances. Endaural occurrences tend to outnumber endonasal occurrences, and a preponderance of both occurs in male children of lower socioeconomic status. The challenge in management of this largely younger target population is to be able to provide reliable, single attempt, atraumatic extraction and avoid the need for general anesthesia, which can occur in 8-10% of cases. Inadequate visualization and access, inappropriately sized instruments, poor patient cooperation, multiple prior attempts with secondary inflammatory reaction, impaction, inexperience, and foreign body consistency and location have been cited as causes for treatment failures.

The three standard methods of foreign body removal include direct instrumentation (ear, nose), irrigation (ear), and suction (ear, nose). Complications have been reported when using direct instrumentation. These complications include cutaneous or mucosal excoriation, abrasion, laceration; bleeding, canal hematoma, otitis externa, facial nerve palsy, iatrogenic tympanic membrane perforation, and aspiration of the foreign body. Direct instrumentation (e.g., Hartman or alligator forceps) can be successfully used for soft objects that present a leading edge or harder, larger objects that will allow placement of a hook or wire loop behind it. For those foreign bodies that are spherical, impacted by occlusion, or lying against the tympanic membrane, such attempts at manipulation can be difficult if not impossible, and potentially dangerous to the patent.

Irrigation affords a relatively atraumatic means for foreign body extraction in the ear canal, particularly in children, but is generally contraindicated with existent tympanic membrane perforations, monomeric tympanic membranes, presence of grommets (relative), hydroscopic or metallic foreign bodies (especially button batteries), and vegetable matter. Additionally, a totally impacted foreign body precludes the beneficial backwash effect of the irrigation solution from dislodging it.

Several techniques for the less traumatic suction extraction of foreign bodies have been described by modifying the end of IV tubing or by affixing a beveled tympanostomy tube within a Frazier suction cannula. While successful in certain cases, they are crude in construction, fail to provide illumination or magnification, and present a limited, non-conformable contact interface.

Other less traditional methodologies have also been reported, e.g., cyanoacrylate contact adhesion, foreign body embedment, and balloon catheter extraction, with similar deficiencies and restrictive applications.

The present invention, as detailed hereinafter, provides a universal instrument that efficiently enables extraction of a foreign object from a body cavity or canal, especially the auditory canal and the endonasal cavity or passage.

### BRIEF DESCRIPTION OF THE DRAWINGS

FIG. 1 is an exploded, perspective view of the extraction device of the present invention; and

FIG. 2 is a view of a suction regulator used herein.

### DETAILED DESCRIPTION OF THE PREFERRED EMBODIMENTS

In accordance with the present invention, and as noted hereinabove, there is provided an extraction device or extractor for removing foreign objects from an external auditory canal, endonasal cavity, or other body passages or cavities where foreign objects can be lodged.

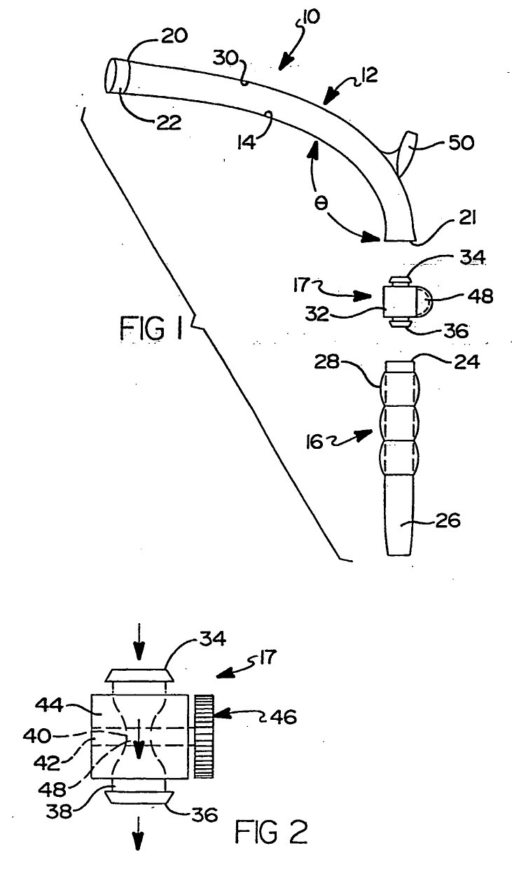

Turning now to the drawings, there is shown in FIG. 1 a preferred embodiment of the extraction device, generally denoted at 10. The extraction device 10 comprises an elongated hollow tubular structure (or suction tube) 12 that is connected to a vacuum or other source of suction (i.e., a source which produces a negative pressure of sufficient magnitude to draw the foreign object against an insertion end of the tube structure), and means 17 for varying or controlling the degree of suction in the tubular structure 12.

The tubular structure 12 includes a first portion 14 and a second portion 16, each portion being elongated, hollow, and generally circular in cross-section. Preferably, each tubular portion 14 and 16 is formed from a non-toxic material, such as a silicone or the like.

The first portion 14 includes a distal end 20, a central body portion 30, and a proximal end 21. Preferably, the opposite ends 20 and 21 of the first portion 14 are frusto-conically shaped (i.e., flare outwardly and increase in diameter) relative to the central body portion 30. The distal end 20 of the first portion 14 is placed within the ear or nose cavity and dimension so as to be in enveloping juxtaposition with the foreign object. The proximal end 21 is removably connected to the means 17 for varying or controlling the degree of suction.

The first portion 14 is curvilinear relative to the central body portion 30. As such, the distal end 20 is in an angular relation with the proximal end 21.

Preferably, the opposite ends 20 and 21 of the first portion 14 are offset and at an angle Θ of about 100° to about 150° to one another. More preferably, the angular offset Θ is about 130° to about 140°.

Further, the first portion 14 is formed from a hard, rigid, non-toxic material, such as silicone, rubber, or the like.

According to an important aspect of this invention, an enveloping lip 22 is removably mounted to the distal end 20 by any suitable means, such as a slip fit or the like. The lip 22 is an annular or toroidal structure that envelops the foreign object (not shown). The lip 22 is preferably formed from a flexible, non-toxic material, such as a silicone rubber, which can be placed over the foreign object. Preferably, frusto-conical lips of different diameter may be mounted to the distal end of the first portion. That is, a lip 22 of predetermined size may be selected for fitment to the distal end of the first portion 14 wherein to accommodate the foreign object, as needed and depending on the patient. So fitted, the distal end 20 of the first portion 14 and the lip 22 fitted thereto form a smooth, continuous, frusto-conical insertion tip.

Also, and according to an aspect of this invention, the lip 22 can be integrally formed with the first portion 14. Under such circumstances, the user would be provided with a plurality of tubular sections, each section being integrally formed with a lip of different diameter.

Referring again to FIG. 1, the second portion 16 of the tubular structure 12 has a distal end 24 operatively connected to the means 17 for varying or controlling the suction pressure, and a proximal end 26 adapted to be removably connected to the source of suction, such as a vacuum or the like (not shown).

As shown in FIG. 1, preferably, the outer surface of the second portion 16 is convoluted at 28. This convolution extends along a section of the second portion 16 and enables easy and secure gripping by virtue of its rubber-like construct.

Preferably, the second portion 16 of the tubular structure 12 is formed from a suitable non-toxic material, such as a rubber, silicone, or the like.

The means 17 for varying or controlling the suction pressure is preferably interposed between the proximal end 21 of the first portion 14 and the distal end 24 of the second portion 16 and enables air to be drawn between the first and second portion 14 and 16.

In a first embodiment thereof, and referring more particularly to FIG. 2, the means 17 for varying and controlling vacuum pressure comprises, in part, a coupler 32. The coupler 32 includes a housing body 44 having opposite ends 34 and 36, and a Venturi passage 38 therethrough, the ends 34 and 36 forming an inlet and outlet to the passage 38. The inlet end 34 is capable of engaging and completing a fluid connection to the proximal end 21 of the first portion 14, such as through a slidable mounting or the like. The outlet end 36 is capable of engaging and completing a fluid connection to the distal end 24 of the second portion 16.

The Venturi passage 38 has a restricted throat 40, in a conventional manner. A valve 42, located within the housing body 44, regulates the degree of opening of the Venturi passage 38 at the restricted throat 40. The valve 42 is in the form of a stem, disposed for rotation in the passage 38, and provided with a central passage 48. The valve 42 functions in a manner similar to a stopcock and the central passage 48 operates to complete a fluid connection between the Venturi passage 38 and the internal passages of the tubular portions 14 and 16.

In a first position, the central passage 48 of the valve stem 42 is in register with the Venturi passage 38, representing a fully open condition. In a second position, the valve stem 42 is rotated 90°, and the central passage 48 of the stem 42 is rotated out of register with the Venturi passage 38, representing a fully closed position wherein the suction source cannot draw the foreign object into and against the insertion lip 22. The amount of rotation of the stem 42 between these two extremes provides a predetermined degree of passage openness between the fully open and fully closed positions and suction force available.

Means 46 for controlling movement of the valve, such as dial 48, is operatively connected to the valve 42.

It is also possible to position the means for controlling suction proximate to the vacuum source.

Also, the housing body 44 can be integral with the tubular structure 12 and configured to house the stopcock valve 42. In this manner, the tubular structure 12 can be assembled as a unitary structure.

A magnifying lens 50, or the like, can be affixed to the first portion 14 to enable the user, such as a physician, to see within the canal or cavity, to ensure that the lip 22 envelops the foreign object to be removed.

In operation, the forward insertion tip or enveloping lip 22 of the extraction device 10 is inserted into the canal or cavity with the lip 22 engaging the foreign object. Thereafter, the suction is applied; the degree of suction being controlled through the means 17 for controlling. As suction is applied, the foreign object is drawn against the enveloping lip 22 and is safely removed.

The present invention provides numerous beneficial features, amongst which are the following:

1. Universal Application—can be used in the ear canal and nasal cavity irrespective of tympanic membrane status, foreign body shape, size or location.

2. Simplicity—prefabricated, handheld instrument requires only visualization of foreign body, tip contact, and vacuum extraction.

3. Adaptability—selection of interchangeable suction heads and control over vacuum pressures optimizes extraction suction with varying foreign body shapes, sizes, and degrees of impaction.

4. Magnification—afforded by suction tube mounted magnifying lens.

5. Patient tolerance—least potentially traumatic method of extraction since foreign body only is engaged rather than manipulated.

6. Cost effective—a single avoidance of intraoperative anesthesia for foreign body removal covers purchase cost twenty-fold.

The present invention provides an efficient and effective means for removing foreign objects from body cavities and canals.

While the present invention has been described with respect to specific embodiments, it will be understood that from the foregoing detailed description and accompanying drawings that various modifications and variations will occur to those skilled in the art. Such modifications and variations are intended to fall within the scope of the appended claims.

What is claimed is:

1. An instrument for extracting foreign bodies from the cavity of a human, wherein the cavity is an auditory canal or endonasal passage of the human, the instrument comprising:
an elongated tubular structure, said tubular structure defining distal and proximal ends and a passageway between the ends,
the distal end portion being generally frusto-conically shaped and positionable within the canal or passage, the frusto-conical end portion including an enveloping lip sized to envelop and be engaged by a foreign body drawn there within, and
the proximal end being removably connectible to a source of negative pressure, a lowering of the pressure in the passageway operating to suction and captivate the foreign particle into the distal end, and
a valve in operable relation with the passageway for adjusting the amount of air that is drawn between the distal and proximal ends of the tubular structure.

2. The instrument as claimed in claim 1, wherein
said valve includes an inlet and an outlet, and
said tubular structure includes first and second portions, said first portion including a rearward end and said distal end, said rearward end being connectible to the inlet of said valve, and said second portion includes a forward end and said proximal end, said forward end being connectible to the outlet of said valve.

3. The instrument as claimed in claim 2, further wherein said first portion is curvilinear and the rearward and distal ends thereof are angularly offset and disposed at an angle Θ relative to one another.

4. The instrument as claimed in claim 3, wherein the angle Θ is about 100° to about 150°.

5. The instrument as claimed in claim 3, wherein the angle Θ is about 130° to about 140°.

6. The instrument as claimed in claim 3, wherein
said valve includes a valve body having said inlet and outlet, a passageway extending between said inlet and outlet and connecting the passages in said first and second portions, and a valve stem having a passageway for varying the amount of flow permitted through the passageway of said valve body and passages of said tube structure.

7. The instrument as claimed in claim 6, wherein said valve stem is mounted for rotation relative to the valve body and positions the passageway thereof in the passageway of said valve body, wherein rotation of the stem causes the passageway in said stem to move into and out of register with the valve passageway and change the amount of air that is permitted to drawn into and through the tubular structure.

8. The instrument as claimed in claim 1, wherein said enveloping lip is removably mounted to said distal end.

9. The instrument as claimed in claim 1, wherein said enveloping lip is integrally formed with the distal end of the first portion.

10. The instrument as claimed in claim 9, wherein said enveloping lip and first portion are formed from a non-toxic material, said lip is of a flexible material, and said first portion is of a hard material.

11. The instrument as claimed in claim 10, wherein said first portion includes a central body portion between said distal and rearward ends, and the cross-section of said first portion expands, outwardly in extending in opposite axial directions from said central body portion towards said distal and proximal ends, and an exterior section of the second portion is provided with convolutions to enable easy and secure gripping.

12. The instrument as claimed in claim 1, further comprising a magnifying lens, the lens being affixed to the first portion to enable the user to see within the cavity and ensure that the lip envelops the foreign object.

13. Apparatus for extracting a foreign body from a cavity of a human, wherein the cavity is an auditory canal or an intranasal passage, the apparatus comprising a hollow generally cylindrical suction tube forming a suction passageway, the suction tube having a distal end that is insertable within the cavity and forms an inlet to the passageway, the distal end being frusto-conically shaped and adapted to be fitted in enclosing relation about the foreign object, a proximal end that forms an outlet from the passageway and is connectible to a source of negative pressure whereby to draw the foreign body into the distal end of the suction tube, and a closure valve located in the passageway and movable between first and second positions to prevent and permit flow through the suction tube.

14. The apparatus as claimed in claim 13, further comprising a magnifying lens, the lens being affixed to the suction tube to enable the user to see within the cavity and ensure that the distal end encloses the foreign object.

15. The apparatus as claimed in claim 13, wherein a forward end portion of the suction tube is curvilinear, the proximal end is connectible to the source of suction, and the closure valve is integrally formed at the proximal end.

16. The apparatus as claimed in claim 13, wherein said suction tube includes first and second tube portions, the first portion being curvilinear and the distal end thereof being insertable in the cavity, the second portion being axially extending and directly connectible to the source of suction, and the closure valve being disposed between the first and second tube portions.

17. The apparatus as claimed in claim 13 wherein the distal end comprises an enveloping lip of a flexible non-toxic material, and the tube is comprised of a generally rigid non-toxic material.

18. The apparatus as claimed in claim 17, wherein the lip is removably attached to the distal end.

19. The apparatus as claimed in claim 17, wherein the lip is integrally formed with the distal end.

20. An extraction instrument for extracting by suction a foreign object from the nasal cavity or ear canal of a human, the instrument comprising:
a curvilinear first tube element, the tube element being generally circular in cross-section and having a forward end, a rearward end, and a central body portion, the forward end being frusto-conically shaped and greater in diameter than the diameter of said central body-portion; the forward end portion forming an insertion tip sized to envelop and capture a foreign object to be extracted from the ear canal or nasal cavity of a human, and the insertion end being angularly offset relative to the rearward end,
said insertion tip and said tube element being formed from a non-toxic material, wherein said tip is of a flexible material and said tube element is of a rigid material,
a second tube element, the element having a rearward end that is connectible to a source of suction and a forward end,
means for controlling and varying the pressure and suction force produced in the tube elements, the means for controlling and varying being interposed between and connected to the rearward and forward ends, respectively, of the first and second tube elements, and
a magnifying glass connected to the first tube element to enable a user to see within the cavity or canal and ensure that the insertion tip seats in enveloping relation about the foreign object.
